(19) United States
(12) Patent Application Publication
(10) Pub. No.: US 2013/0281528 A1
(43) Pub. Date: Oct. 24, 2013

(54) METHOD OF TREATING GASTRIC CANCER

(71) Applicant: Niiki Pharma Acquisition Corp.2, Tampa, FL (US)

(72) Inventor: Hooshmand SHESHBARADARAN, Hoboken, NJ (US)

(21) Appl. No.: 13/655,477

(22) Filed: Oct. 19, 2012

Related U.S. Application Data
(63) Continuation of application No. PCT/US2011/032938, filed on Apr. 19, 2011.
(60) Provisional application No. 61/325,586, filed on Apr. 19, 2010.

(51) Int. Cl. A61K 31/555

(52) U.S. Cl. CPC A61K 31/555
USPC 514/492

(57) ABSTRACT

Methods and compositions for treating gastric cancer are disclosed.

### CROSS-REFERENCE TO RELATED APPLICATIONS

This application is a continuation of PCT/US2011/032938 filed Apr. 19, 2011, which is entitled to the priority of U.S. Provisional Application No. 61/325,586 filed on Apr. 19, 2010.

### FIELD OF THE INVENTION

The present invention generally relates to pharmaceutical compositions and methods for treating cancer, and particularly to a pharmaceutical composition having a ruthenium(III) complex, and method of using thereof.

### BACKGROUND OF THE INVENTION

Gastric cancer is one of the most deadly forms of cancer. Treatment option for gastric cancer has been limited. Surgery and radiation therapy can be used for early-stage gastric cancer, but not very effective for advanced or recurrent gastric cancer. Traditional chemotherapeutic agents such as 5-fluorouracil and cisplatin have shown very limited effect often causing serious side effects. Thus, there is a significant unmet need for new agents and methods for treating gastric cancer.

### SUMMARY OF THE INVENTION

The present invention provides methods of treating gastric cancer. In one aspect, the present invention provides a method of treating, preventing or delaying the onset of, gastric cancer, comprising administering to a patient having gastric cancer a therapeutically or prophylatically effective amount of a pharmaceutically acceptable salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]).

In accordance with another aspect, a method of treating, preventing or delaying the onset of, a refractory gastric cancer is provided comprising administering a therapeutically or prophylatically effective amount of an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) to a patient having refractory gastric cancer.

Use of an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) for the manufacture of a medicament for use in the methods of the present invention is also provided.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### BRIEF DESCRIPTION OF THE DRAWINGS

FIG. 1 is a graph showing the dose-dependent growth inhibition by sodium trans-[tetrachlorobis(1H-indazole)ruthenate(III)] in an MTT assay in gastric cancer cell line NCI-N87. X axis is drug concentration in nM and Y axis is percentage of control in absorbance.

### DETAILED DESCRIPTION OF THE INVENTION

The present invention is at least in part based on the discovery that the compound sodium trans-[tetrachlorobis(1H-indazole)ruthenate(III)] is particularly effective in treating gastric cancer. Accordingly, in accordance with a first aspect of the present invention, a method is provided for treating gastric cancer. The method comprises treating a gastric cancer patient in need of treatment with a therapeutically effective amount of trans-[tetrachlorobis(1H-indazole)ruthenate(III)] or a pharmaceutically acceptable salt thereof. Examples of such a salt include an indazolium salt or alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]). That is, the present invention is directed to the use of an effective amount of a trans-[tetrachlorobis(1H-indazole)ruthenate(III)] or a pharmaceutically acceptable salt thereof (e.g., indazolium or alkali metal salt) for the manufacture of medicaments for treating a gastric cancer in patients identified or diagnosed as having a gastric cancer.

The method of the present invention can be useful in various gastric malignancies including, but not limited to, gastric adenocarcinoma, MALT lymphoma (MALToma), stromal tumors, and gastrointestinal stromal tumor (GIST).

In the various embodiments of this aspect of the present invention, the treatment method optionally also comprises a step of diagnosing or identifying a patient as having gastric tumor. The identified patient is then treated with or administered with a therapeutically effective amount of trans-[tetrachlorobis(1H-indazole)ruthenate(III)] or a pharmaceutically acceptable salt thereof, preferably an alkali metal salt, e.g., sodium trans-[tetrachlorobis(1H-indazole)ruthenate(III)]. Various gastric cancers can be diagnosed in any conventional diagnostic methods known in the art including CT scan, endoscopy, barium roentgenogram, biopsy, etc.

In addition, another aspect of the present invention provides a method of treating refractory gastric cancer comprising treating a patient identified as having refractory gastric cancer with a therapeutically effective amount of trans-[tetrachlorobis(1H-indazole)ruthenate(III)] or a pharmaceutically acceptable salt thereof, preferably an alkali metal salt thereof (e.g., sodium trans-[tetrachlorobis(1H-indazole)ruthenate(III)] or potassium trans-[tetrachlorobis(1H-indazole)ruthenate(III)]).

The term “refractory gastric cancer” as used herein refers to a gastric cancer that either fails to respond favorably to an anti-neoplastic treatment that does not include trans-[tetrachlorobis(1H-indazole)ruthenate(III)] or a pharmaceutically acceptable salt thereof, or alternatively, recurs or relapses after responding favorably to an antineoplastic treatment that does not include trans-[tetrachlorobis(1H-indazole)ruthenate(III)] or a pharmaceutically acceptable salt thereof. Accordingly, “a gastric cancer refractory to a treatment” as used herein means a gastric cancer that fails to respond favorably to, or resistant to, the treatment, or alternatively, recurs or relapses after responding favorably to the treatment.

To detect a refractory gastric cancer, patients undergoing initial treatment can be carefully monitored for signs of resistance, non-responsiveness or recurring gastric cancer. This can be accomplished by monitoring the patient's cancer's response to the initial treatment. The response, lack of response, or relapse of the cancer to the initial treatment can be determined by any suitable method practiced in the art. For example, this can be accomplished by the assessment of tumor size and number. An increase in tumor size or, alternatively, tumor number, indicates that the tumor is not responding to the chemotherapy, or that a relapse has occurred. The determination can be done according to the “RECIST” criteria as described in detail in Therasse et al, J. Natl. Cancer Inst. 92:205-216 (2000).

In accordance with yet another aspect of the present invention, a method is provided for preventing or delaying the onset of gastric cancer, or preventing or delaying the recurrence of gastric cancer, which comprises treating a patient in need of the prevention or delay with a prophylatically effective amount of trans-[tetrachlorobis(1H-indazole)ruthenate(III)] or a pharmaceutically acceptable salt thereof, preferably an alkali metal salt of trans-[tetrachlorobis(1H-indazole)ruthenate(III)] (e g., sodium trans-[tetrachlorobis(1H-indazole)ruthenate(III)] or potassium trans-[tetrachlorobis(1H-indazole)ruthenate(III)]).

For purposes of preventing or delaying the recurrence of gastric cancer, patients with gastric cancer who have been treated and are in remission or in a stable or progression free state may be treated with a prophylatically effective amount of an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) to effectively prevent or delay the recurrence or relapse of gastric cancer.

As used herein, the phrase “treating . . . with . . . ” or a paraphrase thereof means administering a compound to the patient or causing the formation of a compound inside the body of the patient.

In accordance with the method of the present invention, gastric cancer can be treated with a therapeutically effective amount of trans-[tetrachlorobis(1H-indazole)ruthenate(III)] or a pharmaceutically acceptable salt thereof, preferably an alkali metal salt of trans-[tetrachlorobis(1H-indazole)ruthenate(III)], alone as a single agent, or alternatively in combination with one or more other anti-cancer agents.

Alkali metal salts of trans-[tetrachlorobis(1H-indazole)ruthenate(III)] can be made in any methods known in the art. For example, PCT Publication No. WO/2008/154553 discloses an efficient method of making sodium trans-[tetrachlorobis(1H-indazole)ruthenate(III)].

The pharmaceutical compounds such as sodium trans-[tetrachlorobis(1H-indazole)ruthenate(III)] can be administered through intravenous injection or any other suitable means at an amount of from 0.1 mg to 1000 mg per kg of body weight of the patient based on total body weight. The active ingredients may be administered at once, or may be divided into a number of smaller doses to be administered at predetermined intervals of time, e.g., once daily or once every two days. It should be understood that the dosage ranges set forth above are exemplary only and are not intended to limit the scope of this invention. The therapeutically effective amount of the active compound can vary with factors including, but not limited to, the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

In accordance with the present invention, it is provided a use of trans-[tetrachlorobis(1H-indazole)ruthenate(III)] or a pharmaceutically acceptable salt thereof, such as an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]), for the manufacture of a medicament useful for treating gastric cancer. The medicament can be, e.g., in an injectable form, e.g., suitable for intravenous, intradermal, or intramuscular administration. Injectable forms are generally known in the art, e.g., in buffered solution or suspension.

In accordance with another aspect of the present invention, a pharmaceutical kit is provided comprising in a container a unit dosage form of trans-[tetrachlorobis(1H-indazole)ruthenate(III)] or a pharmaceutically acceptable salt thereof, such as an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]), and optionally instructions for using the kit in the methods in accordance with the present invention, e.g., treating, preventing or delaying the onset of gastric cancer, or preventing or delaying the recurrence of gastric cancer, or treating refractory gastric cancer. As will be apparent to a skilled artisan, the amount of a therapeutic compound in the unit dosage form is determined by the dosage to be used on a patient in the methods of the present invention. In the kit, trans-[tetrachlorobis(1H-indazole)ruthenate(III)] or a pharmaceutically acceptable salt thereof such as an alkali metal salt of trans-[tetrachlorobis(1H-indazole)ruthenate(III)] (e.g., sodium trans-[tetrachlorobis(1H-indazole)ruthenate(III)] or potassium trans-[tetrachlorobis(1H-indazole)ruthenate(III)]) can be in lyophilized form in an amount of, e.g., 25 mg, in an ampoule. In the clinic, the lyophilized form can be dissolved and administered to a patient in need of the treatment in accordance with the present invention.

### Example 1

To test the activities of sodium trans-[tetrachlorobis(1H-indazole)ruthenate(III)], ATCC's MTT Cell Proliferation Assay® was performed using human gastric cancer cell line NCI-N87 (differentiated carcinoma). Stock cultures were allowed to grow to 70-80% confluence for this study. The anti-proliferative activity of sodium trans-[tetrachlorobis(1H-indazole)ruthenate(III)], against the indicated cell line was evaluated in vitro using the the ATCC's MTT Cell Proliferation Assay (Catalog No. 30-1010K). NCI-N87 was grown using RPMI1640 (Cell Gro 10-040-CV), with 1% of 1M HEPES, 1% sodium pyruvate, 1% of 45% glucose solution, 10% of heat-inactivated FBS and 1% of pen/strep/glutamine. NCI-N87 cell plates were seeded with 20E+03 cells/well and treated with sodium trans-[tetrachlorobis(1H-indazole)ruthenate(III)] at 1,000 μm, or a series of 4× dilutions thereof (250 μM, 62.5 μM, etc.). 100 μl of medium was removed from each well at 72 hours post-treatment and 10 μl MTT reagent was added to each well. The plates were incubated plate at 37° C. for 4 hours and then 100 μl of detergent was added. The plates were left overnight at room temperature in the dark and was read on a plate reader using SoftMax® Pro (version 5.2, Molecular Devices).

The absorbance data was analyzed as follows: Absorbance values were converted to Percent of Control and plotted against test agent concentrations for IC₅₀ calculations using SoftMax® Pro (version 5.2, Molecular Devices). The plate blank signal average was subtracted from all wells prior to calculating the Percent of Control. Percent of Control values were calculated by dividing the absorbance values for each test well by the No Drug Control average (column 11 values; cells+vehicle control) and multiplying by 100. Plots of Compound Concentration versus Percent of Control were analyzed using the 4-parameter equation to obtain IC₅₀ values and other parameters that describe the sigmoidal dose response curve.

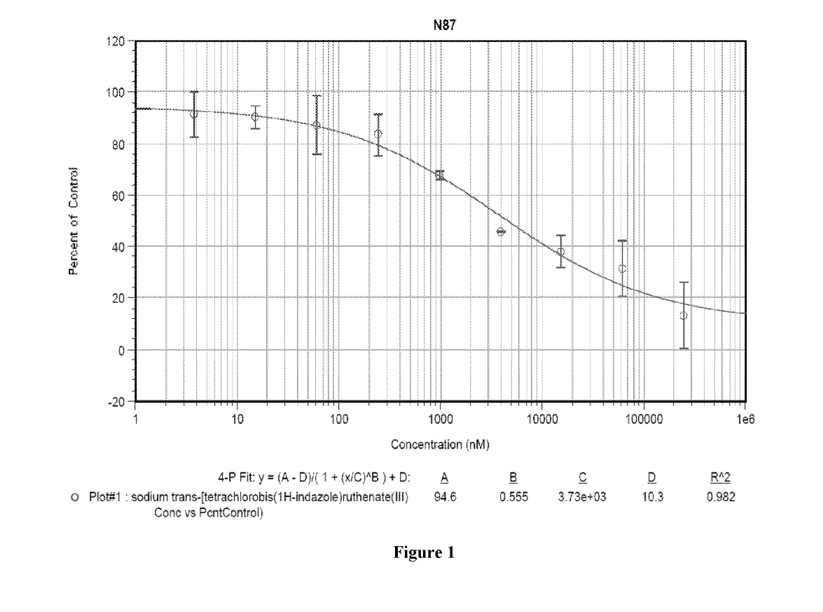

The IC₅₀ value for the test agents was estimated by curve-fitting the data using the following four parameter-logistic equation:

$$Y = \frac{Top - Bottom}{1 + {( X \text{/} {IC}_{50} )}^{n}} + Bottom$$

wherein “Top” is the maximal % of control absorbance (100%) (Value “A” in FIG. 1), “Bottom” is the minimal % of control absorbance at the highest agent concentration (down to zero) (Value “D” in FIG. 1), Y is the Percent of Control absorbance, X is the test agent Concentration, IC₅₀ is the concentration of agent that inhibits cell growth by 50% compared to the control cells (Value “C” in FIG. 1), n is the slope of the curve (Value “B” in FIG. 1). The IC₅₀ of sodium trans-[tetrachlorobis(1H-indazole)ruthenate(III)] in NCI-N87 cell line was 3.73 μM.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be apparent that certain changes and modifications may be practiced within the scope of the appended claims.

What is claimed is:

1. A method of treating gastric cancer, comprising:
identifying a patient having gastric cancer; and
treating the patient with a therapeutically effective amount of a pharmaceutically acceptable salt of trans-[tetrachlorobis(1H-indazole)ruthenate(III)].

2. The method of claim 1, wherein said salt is an alkali metal salt of trans-[tetrachlorobis(1H-indazole)ruthenate(III)].

3. The method of claim 1, wherein said pharmaceutically acceptable salt of trans-[tetrachlorobis(1H-indazole)ruthenate(III)] is sodium trans-[tetrachlorobis(1H-indazole)ruthenate(III)]).
